(19) United States
(12) Patent Application Publication
(10) Pub. No.: US 2010/0099999 A1
(43) Pub. Date: Apr. 22, 2010

(54) ORAL END TIDAL CARBON DIOXIDE PROBE

(76) Inventors: Anna R. Hemnes, Nashville, TN (US); Alexander Newman, Nashville, TN (US); John Newman, Nashville, TN (US)

Correspondence Address: STRATEGIC PATENTS P.C.., C/O PORTFOLIOIP, P.O. BOX 52050, MINNEAPOLIS, MN, 55402, US

(21) Appl. No.: 12/578,841

(22) Filed: Oct. 14, 2009

Related U.S. Application Data
(60) Provisional application No. 61/106,066, filed on Oct. 16, 2008.

(51) Int. Cl. A61B 5/08

(52) U.S. Cl. 600/532

(57) ABSTRACT

In embodiments of the present invention improved capabilities are described for evaluating pulmonary embolism. A system and method of evaluating pulmonary embolism in a subject may include measuring end tidal partial pressure of exhaled carbon dioxide in the subject, wherein the measurement is made orally, obtaining a clinical approximation of dead space ventilation based on the measurement, and excluding pulmonary embolism when the end tidal partial pressure of exhaled carbon dioxide reaches a threshold.

This application claims the benefit of U.S. Patent Application No. 61/106,066, filed Oct. 16, 2008, the entire disclosure of which is herein incorporated by reference.

### BACKGROUND

1. Field

The present invention relates to an oral end tidal carbon dioxide probe.

2. Description of the Related Art

Pulmonary embolism (PE) remains a diagnostic challenge and many studies are performed with a low yield at substantial financial cost and potential risk from radiation. End tidal carbon dioxide (EtCO₂) is a surrogate for pulmonary vascular obstruction and subsequent dead space ventilation. Using EtCO₂ as an initial screening test in patients being evaluated for PE would potentially spare many unnecessary, low-yield diagnostic studies and their associated risk and financial burden.

Pulmonary embolism (PE) is a common concern in the evaluation of diverse clinical presentations including chest pain, dyspnea and hypoxemia. Extensive diagnostic evaluation, including contrast enhanced helical computed tomography (CT), is frequently undertaken, despite a relatively low incidence of disease, as described for example in DeMonaco N A, Dang Q, Kapoor W N, Ragni M V., Pulmonary embolism incidence is increasing with use of spiral computed tomography, Am J Med 2008: 121(7): 611-617 which is incorporated herein by reference in its entirety. In addition to the cost of these studies, the risks of contrast and radiation exposure add to the burden of evaluation, as described for example in Parfrey P S, Griffiths S M, Barrett B J, Paul M D, Genge M, Withers J, Farid N, McManamon P J., Contrast material-induced renal failure in patients with diabetes mellitus, renal insufficiency, or both, A prospective controlled study, N Engl J Med 1989: 320(3): 143-149 and Brenner D J, Hall E J., Computed tomography—an increasing source of radiation exposure, N Engl J Med 2007: 357(22): 2277-2284 each of which is incorporated herein by reference in its entirety.

Diagnostic algorithms to simplify testing procedures in PE diagnosis have been explored, most combining D-dimer testing and CT angiography, as described for example in Di Nisio M, Squizzato A, Rutjes A W, Buller H R, Zwinderman A H, Bossuyt P M., Diagnostic accuracy of D-dimer test for exclusion of venous thromboembolism: a systematic review, J Thromb Haemost 2007: 5(2): 296-304 and Wells P S, Anderson D R, Rodger M, Stiell I, Dreyer J F, Barnes D, Forgie M, Kovacs G, Ward J, Kovacs M J., Excluding pulmonary embolism at the bedside without diagnostic imaging: management of patients with suspected pulmonary embolism presenting to the emergency department by using a simple clinical model and d-dimer, Ann Intern Med 2001: 135(2): 98-107 each of which is incorporated herein by reference in its entirety. D-dimer testing requires venipuncture and time for test performance, as described for example in Tapson V F., Acute pulmonary embolism, N Engl J Med 2008: 358(10): 1037-1052 and DiNisio as referenced above each of which is incorporated herein by reference in its entirety. CT angiography use in PE diagnosis has increased markedly, as described in DeMonaco referenced above. As a low percentage of CT angiograms demonstrate PE, as described for example in DeMonaco referenced above, Perrier A, Roy P M, Sanchez O, Le Gal G, Meyer G, Gourdier A L, Furber A, Revel M P, Howarth N, Davido A, Bounameaux H., Multidetector-row computed tomography in suspected pulmonary embolism, N Engl J Med 2005: 352(17): 1760-1768, and Stein P D, Fowler S E, Goodman L R, Gottschalk A, Hales C A, Hull R D, Leeper K V, Jr., Popovich J, Jr., Quinn D A, Sos T A, Sostman H D, Tapson V F, Wakefield T W, Weg J G, Woodard P K. Multidetector computed tomography for acute pulmonary embolism, N Engl J Med 2006: 354(22): 2317-2327 each of which is incorporated herein by reference in its entirety, concern has been raised of the contrast and radiation risk, as described for example in Brenner referenced above and Amis E S, Jr., Butler P F, Applegate K E, Birnbaum S B, Brateman L F, Hevezi J M, Mettler F A, Morin R L, Pentecost M J, Smith G G, Strauss K J, Zeman R K., American College of Radiology white paper on radiation dose in medicine, J Am Coll Radiol 2007: 4(5): 272-284 each of which is incorporated herein by reference in its entirety. Clinical prediction rules, including the Wells score, have also been proposed which have the advantage of instantaneous results, avoidance of invasive procedures, and low risk and cost, as described for example in Wells referenced above and Miniati M, Bottai M, Monti S, Salvadori M, Serasini L, Passera M., Simple and accurate prediction of the clinical probability of pulmonary embolism, Am J Respir Crit Care Med 2008: 178(3): 290-294 each of which is incorporated herein by reference in its entirety. Thus there is a need for a safer, more accurate and readily available diagnostic testing for PE.

### SUMMARY

The D-dimer test has been studied extensively in the exclusion of PE and its value in exclusion of low risk patients for further diagnostic evaluation is well established, as described for example in Tapson referenced above. Despite a high negative predictive value in low risk patients, the D-dimer test has a highly variable sensitivity and its interpretation can be confusing with multiple commercially available tests and cut-off values, as described for example in Stein P D, Hull R D, Patel K C, Olson R E, Ghali W A, Brant R, Biel R K, Bharadia V, Kalra N K., D-dimer for the exclusion of acute venous thrombosis and pulmonary embolism: a systematic review, Ann Intern Med 2004: 140(8): 589-602 and Siragusa S, Terulla V, Pirrelli S, Porta C, Falaschi F, Anastasio R, Guarnone R, Scarabelli M, Odero A, Bressan M A., A rapid D-dimer assay in patients presenting at the emergency room with suspected acute venous thrombosis: accuracy and relation to clinical variables, Haematologica 2001: 86(8): 856-861 each of which is incorporated herein by reference in its entirety. Most importantly, D-dimer testing requires venipuncture and time for transport, measurement and reporting which may increase total healthcare expenditure. A more rapidly available test would enhance speed of decision-making.

End tidal carbon dioxide (EtCO₂) level measurement is a physiological surrogate for diagnosing vascular obstruction resulting from PE. Pulmonary thromboembolism results in dead space ventilation and therefore prevents meaningful gas exchange in the subtended lung unit, yielding an alveolar CO₂ content as low as zero mmHg. As a result, carbon dioxide content measured at end expiration, which represents admixture of all alveolar gas, drops in proportion to dead space ventilation. While there are many potential etiologies of increased dead space ventilation including advanced chronic obstructive pulmonary disease, these diseases are usually easily identified. Increased dead space ventilation is not associated with common clinical conditions that can present similarly to pulmonary embolism e.g. unstable angina, gastroesophageal reflux. Dead space measurement and arterial-alveolar carbon dioxide tension gradient have been studied in the evaluation of PE as described for example in Kline J A, Meek S, Boudrow D, Warner D, Colucciello S., Use of the alveolar dead space fraction (Vd/Vt) and plasma D-dimers to exclude acute pulmonary embolism in ambulatory patients, Acad Emerg Med 1997: 4(9): 856-863 and Rodger M A, Bredeson C N, Jones G, Rasuli P, Raymond F, Clement A M, Karovitch A, Brunette H, Makropoulos D, Reardon M, Stiell I, Nair R, Wells P S., The bedside investigation of pulmonary embolism diagnosis study: a double-blind randomized controlled trial comparing combinations of 3 bedside tests vs ventilation-perfusion scan for the initial investigation of suspected pulmonary embolism, Arch Intern Med 2006: 166(2): 181-187 each of which is incorporated herein by reference in its entirety, but the utility of end tidal CO₂ measurement alone in diagnosis of pulmonary embolism is not known. EtCO₂ is safe, non-invasive, inexpensive, and rapidly done at the bedside, whereas dead space measurement requires collection of exhaled gas and alveolar-arterial gradient requires arterial blood gas sampling.

In an aspect of the invention, a system and method of evaluating pulmonary embolism in a subject may include measuring carbon dioxide content at end expiration to obtain the end tidal partial pressure of exhaled carbon dioxide in the subject, wherein the measurement is made orally, obtaining a clinical approximation of dead space ventilation based on the measurement, and excluding pulmonary embolism when the end tidal partial pressure of exhaled carbon dioxide reaches a threshold. In the method and system, the threshold is at least 36 mm Hg. The method and system may further include applying a clinical prediction rule. The rule may include calculating a Wells score, and pulmonary embolism may be excluded when the Wells score is at least four. In the method and system, the subject may be a pediatric subject. In the method and system, the subject may be sedated. In the method and system, the subject may be intubated.

In an aspect of the invention, an oral capnometer may include an oral gas capture member, for collecting expired gases from the mouth, and a carbon dioxide measuring device attached to the oral gas capture member for determining levels of expired carbon dioxide from the mouth of a subject. In the method and system, the subject may be a pediatric subject. In the method and system, the subject may be sedated. In the method and system, the subject may be intubated. In the method and system, carbon dioxide levels may be measured continuously. In the method and system, the expired carbon dioxide may be end tidal carbon dioxide.

In an aspect of the invention, a method of measuring end tidal carbon dioxide in a subject may include collecting expired gases from the mouth through an oral gas capture member adapted to be disposed on the sampling input of a carbon dioxide measuring device and a carbon dioxide measuring device attached to the oral gas capture member for determining levels of expired carbon dioxide from the mouth of the subject. In another aspect of the invention, a method of measuring end tidal carbon dioxide in a subject may include a carbon dioxide measuring device that directly collects expired gases from the mouth of the subject by means of an integral gas capture chamber. In the method and system, the subject may be a pediatric subject. In the method and system, the subject may be sedated. In the method and system, the subject may be intubated. In the method and system, the subject may be awake. In the method and system, the subject may be spontaneously breathing. In the method and system, carbon dioxide levels may be measured continuously. In the method and system, the expired carbon dioxide may be end tidal carbon dioxide.

In an aspect of the invention, a system and method may comprise an oral gas capture member, for collecting expired gases from the mouth of a subject; a gas sensor for identifying and measuring at least one exhaled gas; and a housing for housing the gas sensor, wherein the housing is integral with the oral gas capture member. In the system and method, the exhaled gas may be at least one of carbon dioxide, carbon monoxide, nitrogen, oxygen, and ketone. In the system and method, the subject may be at least one of awake, spontaneously breathing, pediatric, sedated, intubated, sleeping, and the like. In the system and method, gas levels may be measured continuously. In the system and method, the expired carbon dioxide may be end tidal carbon dioxide. In the system and method, the gas sensor may also the measure pH of an exhaled gas.

These and other systems, methods, objects, features, and advantages of the present invention will be apparent to those skilled in the art from the following detailed description of the preferred embodiment and the drawings. All documents mentioned herein are hereby incorporated in their entirety by reference.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The invention and the following detailed description of certain embodiments thereof may be understood by reference to the following figures:

FIG. 1 depicts an image of the modified capnometer of the invention.

FIG. 2 depicts a study flow diagram.

FIG. 3 depicts end tidal carbon dioxide in normal volunteers, patients without pulmonary embolism, and patients with pulmonary embolism.

FIG. 4 depicts end tidal carbon dioxide performance characteristics and pulmonary embolism diagnosis.

FIG. 5 depicts the oral gas capture member of the invention.

FIG. 6 depicts the invention in which the gas capture chamber forms an integral part of the capnometer.

FIG. 7 depicts the invention in which the gas capture chamber is detachably attached to the capnometer, such that other measuring devices may be attached to the gas capture chamber.

FIG. 8 depicts a flow chart of a method for excluding pulmonary embolism.

FIG. 9 depicts a flow chart of a method of measuring end tidal carbon dioxide in a subject.

### DETAILED DESCRIPTION

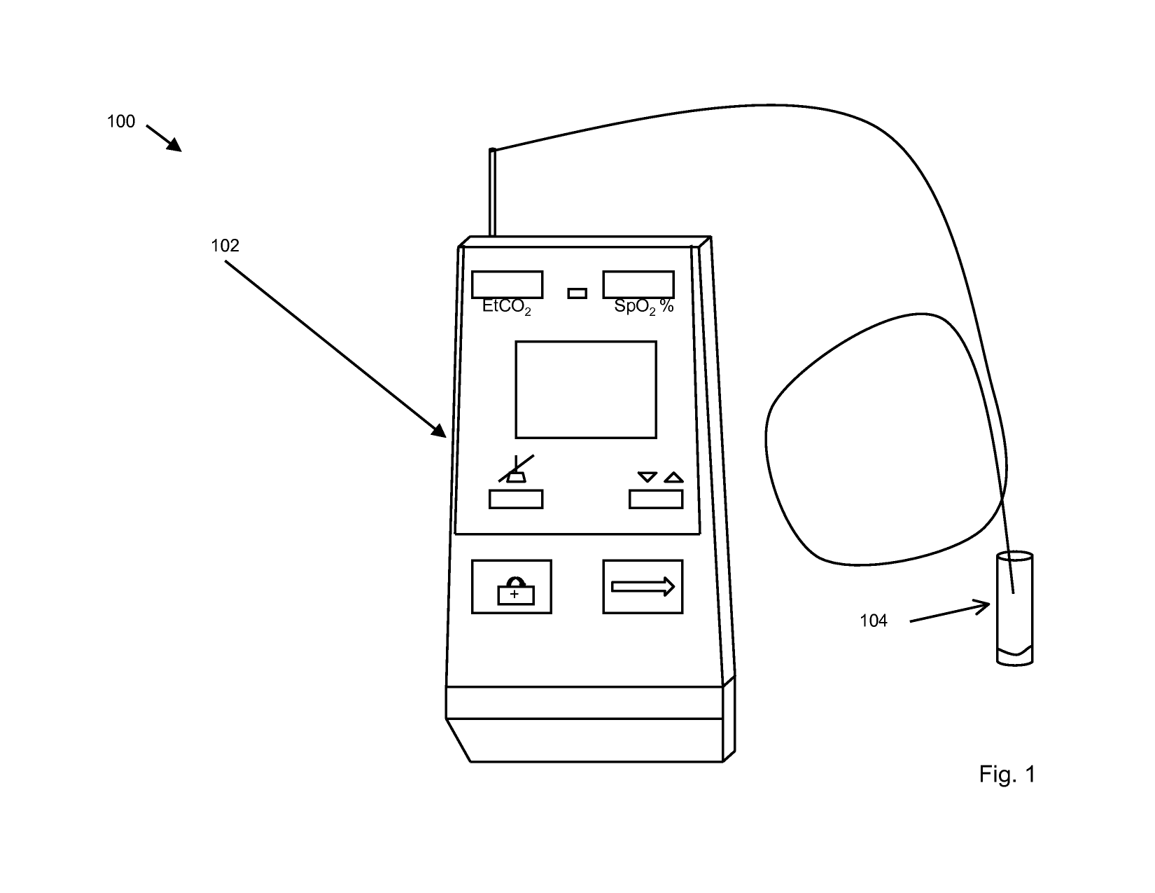

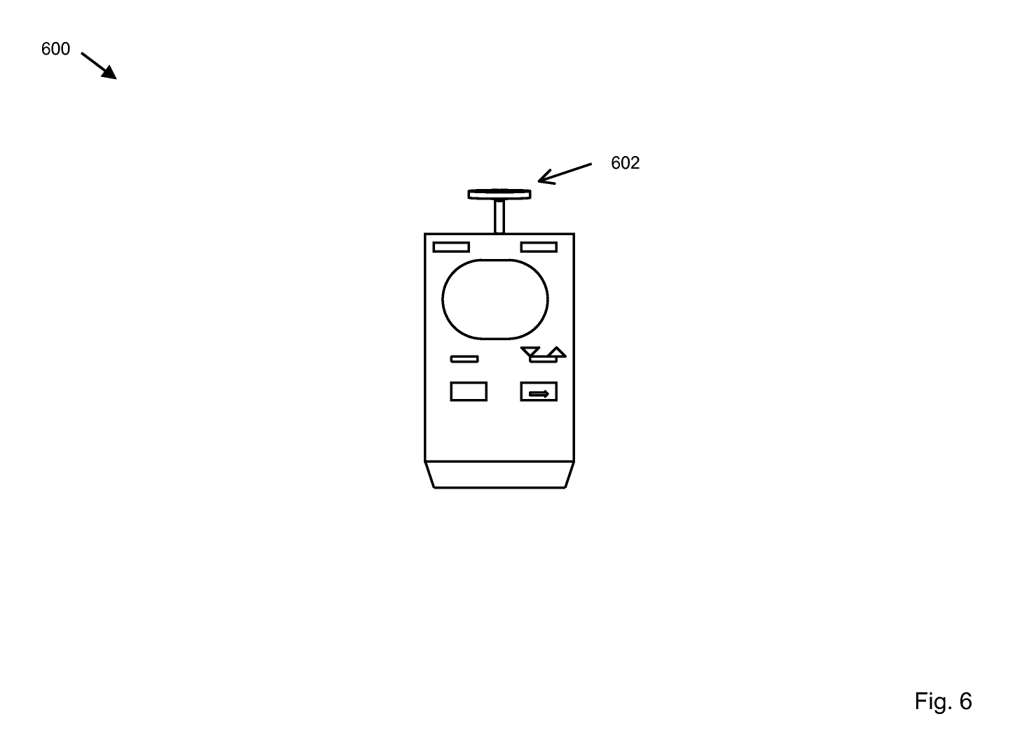

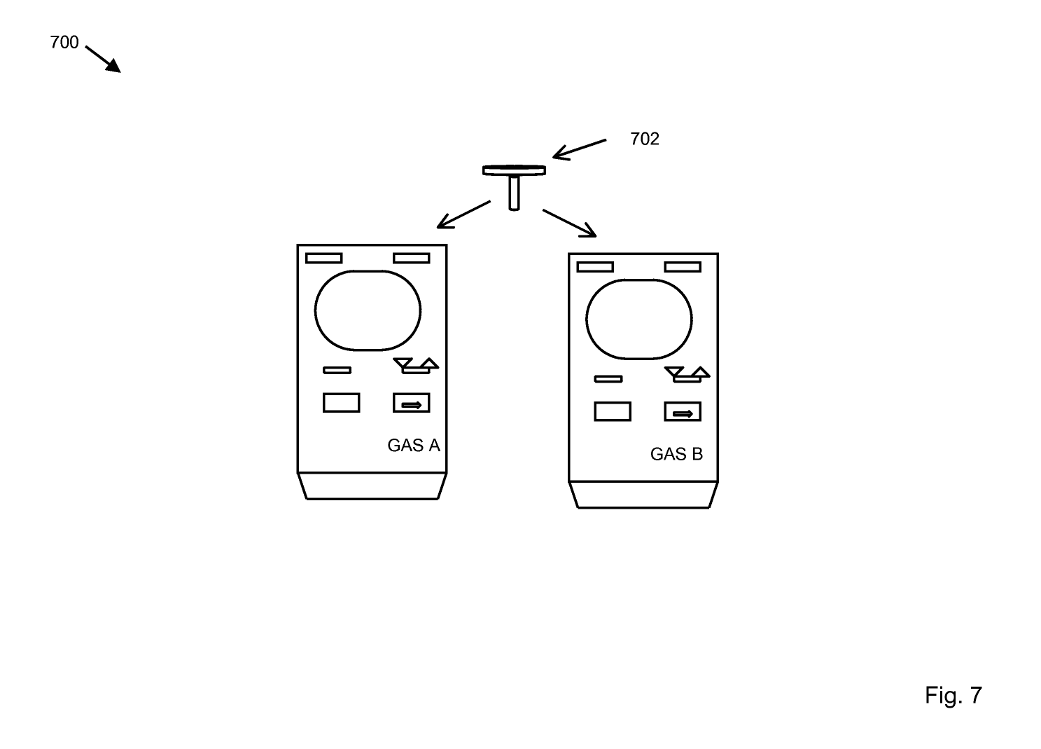

The present invention is an oral capnometer 102 for measuring end tidal carbon dioxide content as it is exhaled from the mouth. Sampling orally exhaled gases may comprise using a capnometer or capnograph with an adaptor on the sampling input to enable oral sampling, as in FIG. 1, an integral oral gas capture member as in FIG. 6, or a detachably engaged oral gas capture member as in FIG. 7. For example, the oral capnometer 102 may be attached to plastic tubing with an adapter that is placed in the mouth. The adapter may be sized to sample gases exhaled from the oral cavity. In other embodiments, the present invention may be an integral oral gas capture member 602 in which the capturing space is connected integrally to the capnometer. In still other embodiments, the oral sampling space may be interchangeably attached to the capnometer to facilitate measurements of exhaled gasses from subjects of various sizes or states of health. Sampling gases from the mouth instead of the nose enables more accurate measurement of exhaled gases as nasal sampling may cause hyperventilation. For example and without limitation, oral sampling of exhaled gases may enable more accurate measurements of end tidal carbon dioxide (EtCO₂), and therefore, more accurate estimation of dead space ventilation.

By measuring EtCO₂2 in patients undergoing evaluation for PE without controlling clinical care or management, predictions may be made regarding PE status. For example, EtCO₂ may be reduced in patients with PE and a normal EtCO₂ measurement may have a high negative predictive value to exclude PE.

The oral capnometer 102 may also be useful for measuring exhaled oxygen levels, such as for estimating cardiac output or other metabolic equivalents. The oral capnometer 102 may also be useful for measuring exhaled carbon monoxide levels, such as in the detection of ongoing cigarette smoking, carbon monoxide poisoning, and the like. The oral capnometer 102 may also be useful for measuring exhaled residual compounds left in the lungs to aid in the diagnosis of some cancers. The oral capnometer 102 may also be useful for measuring exhaled ketones, such as in the diagnosis of ketoacidosis. The oral capnometer 102 may also be useful for measuring the pH of exhaled gas for diagnosis of metabolic acidosis in lactic acidosis or diabetic ketoacidosis. The oral capnometer 102 may also be useful for measuring exhaled nitrogen. The oral capnometer 102 may comprise a gas sensor that is capable of measuring many different gases and pH levels. Alternatively, each gas may be sensed by an individual gas sensor housed separately. Thus, the oral gas capture member 702 may be detachably associated, as shown in FIG. 7 for two devices measuring “Gas A” and “Gas B”, with the oral capnometer 102 such that if measurement of a gas with another gas sensing device is required, the oral gas capture member may be attached to and used with the device. In an embodiment, multiple sizes and shapes of oral gas capture members, suitable for subjects of different ages, sizes and physical conditions, may be detachably attached to the oral capnometer 102.

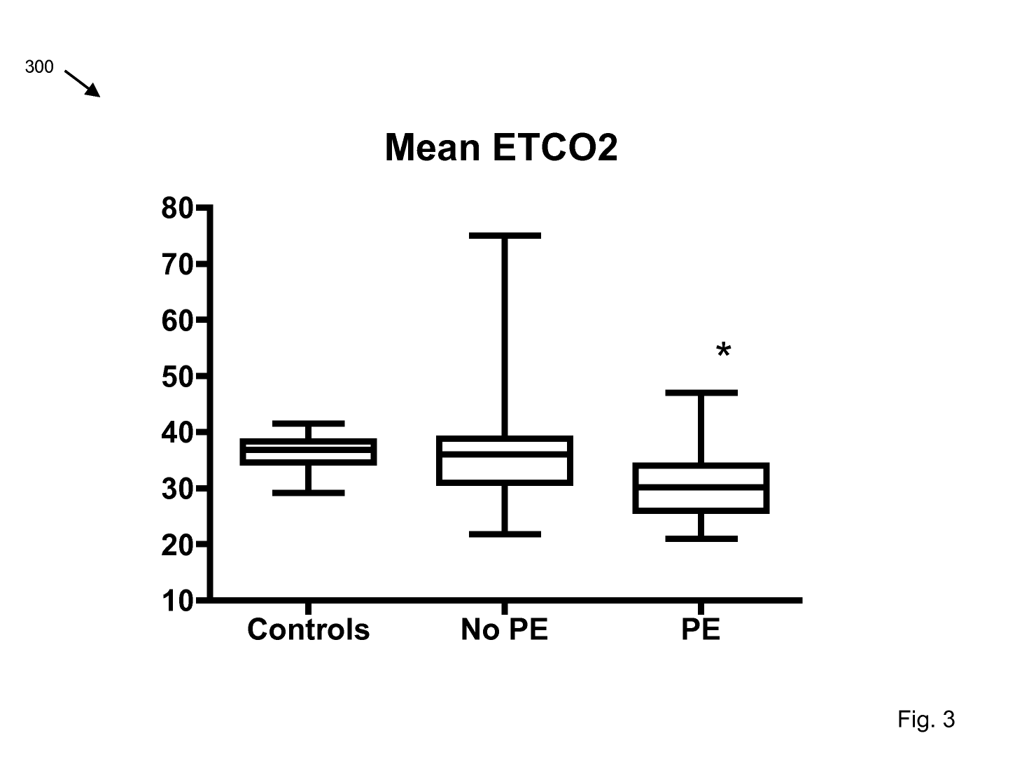

In order to demonstrate the usefulness of accurate end tidal carbon dioxide sampling, the oral capnometer 102 of the invention was used in defining the optimal end tidal carbon dioxide (EtCO₂) level in the exclusion of pulmonary embolism (PE) in patients undergoing evaluation of possible thromboembolism. The oral capnometer 102 of the invention was used in a study involving 298 patients conducted over 6 months at a single academic center. EtCO₂ was measured within 24 hours of contrast enhanced helical CT, lower extremity duplex or ventilation/perfusion scan. Performance characteristics were measured by comparing test results with clinical diagnosis of PE. The results of the study using the oral capnometer 102 were that PE was diagnosed in 39 patients (13%). FIG. 3 depicts mean end tidal carbon dioxide±SD in healthy volunteers, patients without pulmonary embolism (no pulmonary embolism) and patients with pulmonary embolism (pulmonary embolism). The data had a p<0.05 vs. healthy volunteers and no pulmonary embolism group. The mean EtCO₂ in the healthy volunteers was not different from EtCO₂ in the enrolled patients without PE (36.3±2.8, SD mmHg vs. 35.5±6.8 mmHg), as shown in FIG. 3. EtCO₂ in the patients with PE was 30.5±5.5 mmHg (p<0.001 versus no PE group). EtCO₂ of ≧36 mmHg had optimal sensitivity and specificity (87.2 and 53.0% respectively) with a negative predictive value of 96.6% (92.3-98.5 95% CI). This increased to 97.6% (93.2-99.2 95% CI) when combined with a Wells score <4. EtCO₂ of >36 mmHg may reliably exclude PE. Accuracy is augmented by combination with a Wells score. EtCO₂ may be prospectively compared to D-dimer in accuracy and simplicity to exclude PE.

All patients ≧18 years of age who were seen in the Emergency Department or inpatient wards at an academic university hospital over the six month period were screened electronically for a computer order for contrasted chest helical CT, ventilation-perfusion lung scan, pulmonary angiogram or lower extremity Duplex evaluation. Patients meeting screening criteria were approached for consent to undergo EtCO₂ within 24 hours of study order placement. Exclusion criteria were inability to consent, pregnancy, known hypercarbic respiratory failure, mechanical ventilation, face mask oxygen or more than 5 L/minute nasal cannula oxygen or known neuromuscular disease. Patients who presented for evaluation more than once could be enrolled multiple times (n=5, two studies each).

EtCO₂ was measured by a trained single tester blinded to diagnosis using the oral capnometer 102 of the invention, as described for example in Manual O. Operators Manual, NPB 75: Portable bedside capnograph/pulse oximeter. Nellcor Puritan Bennet, Pleasonton, Calif., 1998 which is incorporated herein by reference in its entirety. The device may be calibrated to ±2 mmHg up to 38 mmHg and ±0.08% for every 1 mmHg over 40 mmHg. The oral capnometer 102 is different from capnometers used to measure exhalation from the nostrils in that the uptake cannula is inserted into a plastic tube that, when placed in the mouth, may enable patients to tidally breathe while CO₂ is measured, as shown in FIG. 1. CO₂ Patients were instructed to breathe normally and were tested for five breaths in either a supine or seated position. Nostrils were not clipped shut. EtCO₂ for each breath and respiratory rate were measured. The oral capnometer 102 of the invention was validated every two weeks at two levels of CO₂ using an exercise machine calibrated to zero and 5.6% CO₂. Patient charts were analyzed for demographic data including comorbid conditions and thromboembolic risks, self-reported race/ethnicity (categorized into Hispanic, African-American, Caucasian, or other) results of serum chemistries, blood counts, ventilation/perfusion lung scan, CT (such as Brilliance CT 64 Channel, Phillips, Amsterdam, The Netherlands), pulmonary angiography, and venous duplex exams. Wells score, as described for example in Wells referenced above, was assigned by a single physician, blinded from final diagnosis, from data obtained at the time that diagnostic tests were ordered. Plasma D-dimer testing (STA LIATEST, Diagnostica Stago, Parsippany, N.J., as described for example in Lehman C M, Wilson L W, Rodgers G M., Analytic validation and clinical evaluation of the STA LIATEST immunoturbidimetric D-dimer assay for the diagnosis of disseminated intravascular coagulation, Am J Clin Pathol 2004: 122(2): 178-184 which is incorporated herein by reference in its entirety) was performed at the discretion of the treating physician. Patients with D-dimer testing alone for PE were not included in this study because of the risk of false positive D-dimer tests.

Pulmonary embolism was defined by a published consensus criteria, as described for example in Tapson referenced above, including positive contrast-enhanced CT, intermediate or high probability ventilation perfusion lung scan (as described for example in PIOPED I., Value of the ventilation/perfusion scan in acute pulmonary embolism, Results of the prospective investigation of pulmonary embolism diagnosis (PIOPED), The PIOPED Investigators, JAMA 1990: 263(20): 2753-2759 which is incorporated herein by reference in its entirety) combined with high pretest probability, or positive lower extremity duplex examination with a high clinical suspicion for PE.

To ensure accuracy and reproducibility, and to standardize the modified sensing device, and discover stability of EtCO₂ measurements over time in healthy individuals, EtCO₂ was measured for five breaths in 24 healthy volunteers (mean age 40.0 (12.0), 10/24 male) on three different days. Additionally, EtCO₂ was measured with different FiO₂ delivered by nasal cannula up to 5 lpm and found no difference (data not shown).

Based on the study center's experience and previous work, as described for example in Stein referenced above and Kline J A, Israel E G, Michelson E A, O′Neil B J, Plewa M C, Portelli D C., Diagnostic accuracy of a bedside D-dimer assay and alveolar dead-space measurement for rapid exclusion of pulmonary embolism: a multicenter study, JAMA 2001: 285(6): 761-768 which is incorporated herein by reference in its entirety, a 15% positive rate of diagnostic tests for patients undergoing PE evaluation was assumed. Given this diagnostic rate and a standard deviation of 2.8 mmHg in EtCO₂ measurements in normal volunteers, a sample size calculation determined that 300 patients would be required to detect a difference in EtCO₂ of 1.3 mmHg between groups with 80% power at an alpha level of 0.05. This sample size would allow detection of a difference of 9% in sensitivity compared to the Wells score <4, as described for example in Wells referenced above. Continuous variables are reported as mean (standard deviation) and analyzed using Student's t-test or Wilcoxon Rank Sum testing. Categorical variables are reported as percentages and were analyzed using Fisher's Exact test. Receiver Operating Characteristic (ROC) curves with area under the curve (AUC) were used for determining the optimal EtCO₂ to discriminate between patients with and without PE. All p-values are two-tailed and values ≦0.05 were considered significant. Data analyses were done using both R version 2.7.1 and SPSS (Version 15.0; Chicago, Ill., USA).

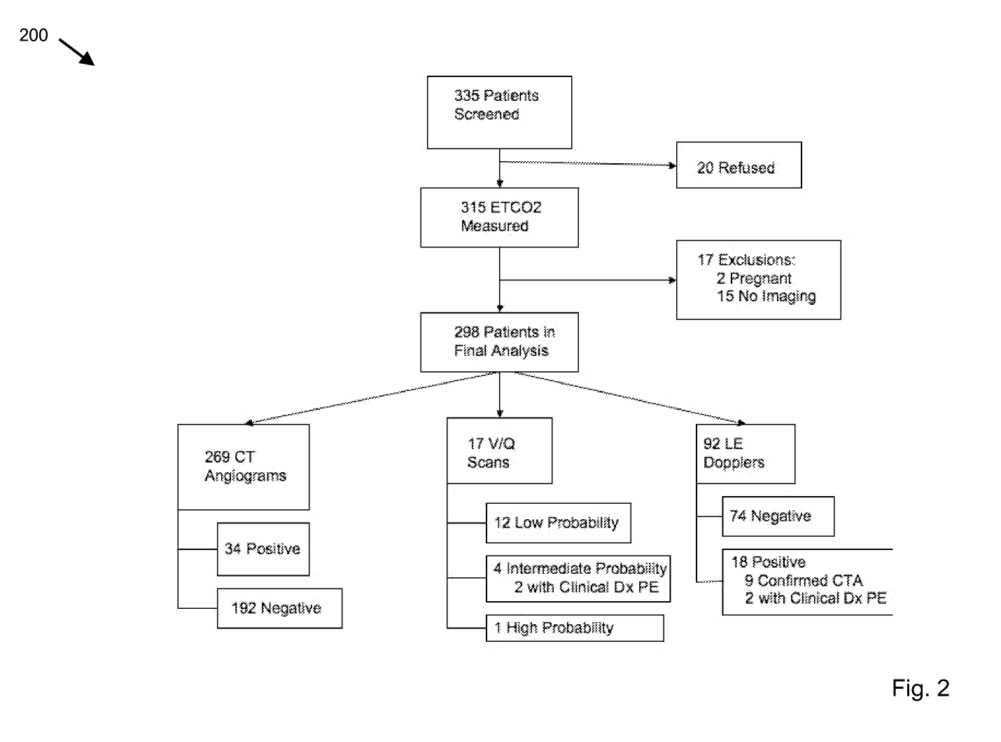

Referring to FIG. 2, a study flow diagram 200 is shown. The study flow diagram 200 shows that a total of 335 patients were screened and approached for entry into the trial. Twenty patients did not consent. Of the 315 patients in whom EtCO₂ was measured, 17 patients were excluded after enrollment (two were found to be pregnant and 15 did not have any imaging studies, as in FIG. 2. Of the remaining 298 patients included in the final analysis, 39 were diagnosed with pulmonary embolism (34 positive helical CT, three intermediate or high probability ventilation perfusion scans with high clinical suspicion, two positive lower extremity duplex examinations with high clinical suspicion). Five patients were enrolled twice. One hundred eighty patients were enrolled from the Emergency Department with 21 PEs and 118 were inpatients with 18 PEs.

Demographic characteristics of the group as a whole and the sub-categories of those with and without PE are shown in Table 1 (Data are presented as mean±SD unless otherwise stated, n=298 unless otherwise stated, p values are for No PE vs. PE groups.)

TABLE 1
Demographics

| | | No PE | | |
|---|---|---|---|---|
| | All (n = 298) | (n = 259) | PE (n = 39) | p Value |
| Age (yrs) | 52.1 ± 17.2 | 51.0 ± 17.1 | 59.5 ± 16.05 | 0.004 |
| Gender (% female) | 53 | 54 | 46 | 0.36 |
| Race (%, n = 294) | | | | |
| White | 72 | 72 | 77 | |
| African-American | 25 | 25 | 23 | |
| Other | 3 | 3 | 0 | |
| Smoking | | | | |
| (%, n = 290) | | | | |
| Never | 53 | 53 | 54 | 0.39 |
| Current | 32 | 33 | 24 | |
| Past | 15 | 14 | 22 | |
| Comorbidities (%) | | | | |
| None | 33 | 33 | 31 | 0.17 |
| Diabetes | 3 | 2 | 10 | |
| Hypertension | 25 | 25 | 23 | |
| Diabetes + | 13 | 14 | 8 | |
| hypertension | | | | |
| Cancer | 13 | 12 | 15 | |
| Chronic lung | 6 | 7 | 3 | |
| disease | | | | |
| Other | 7 | 7 | 10 | |
| PE Risk Factors (%) | | | | |
| None | 62 | 68 | 18 | <0.001 |
| Post-operative | 4 | 4 | 5 | |
| Cancer | 13 | 12 | 18 | |
| Post-partum | 1 | 1 | 0 | |
| Immobilized | 3 | 2 | 8 | |
| Previous DVT/PE | 8 | 7 | 13 | |
| Multiple | 8 | 4 | 33 | |
| Other | 1 | 0 | 5 | |

There was no difference in age, gender, ethnicity, smoking status or presence or absence of medical comorbidities in the two groups. The group with PE was significantly enriched for the presence of one or more risk factors for venous thromboembolic disease than the no PE group (p<0.001). The group without PE had a range of diagnoses from no cause identified (n=44, 17%), pulmonary disease such as COPD, asthma or lung cancer (n=84, 32%), and cardiac disease (n=48, 19%) to musculoskeletal disease, neuromuscular disease, and deep venous thrombosis without PE which made up the remainder.

Patients with PE were less likely than those without PE to undergo chest CT imaging for chest pain alone (p=0.01 PE vs. No PE groups, Table 2), however there were no significant differences in the other indications for chest imaging between the two groups. (Data are presented as mean±SD unless otherwise stated, n=298 unless otherwise stated, p values are for No PE vs. PE groups.) The mean Wells score was 4.3±2.5 in the group with PE and 1.7±1.9 (p<0.001) in the no PE group. Five of 39 patients with PE had a Wells score ≦2.0. Fourteen percent of CTs in the emergency department were positive for PE and 17% of CTs ordered as an inpatient were positive for PE. 97/298 patients had serum D-dimer measured, of these 47 were negative (0 PEs) and 48 positive (4 PEs).

TABLE 2
Presenting Features of Study Enrollees

| | All (n = 298) | No PE (n = 259) | PE (n = 39) | p Value |
|---|---|---|---|---|
| Indication for PE | | | | |
| evaluation (%) | | | | |
| Chest pain | 35 | 37 | 23 | 0.006 |
| Hypoxemia | 1 | 0 | 5 | |
| Dyspnea | 25 | 24 | 31 | |
| Hemoptysis | 0 | 0 | 3 | |
| Fever | 6 | 6 | 5 | |
| Chest pain and | 9 | 8 | 15 | |
| dyspnea | | | | |
| Limb swelling/pain | 4 | 4 | 3 | |
| Miscellaneous | 20 | 21 | 15 | |
| Wells score | 2.0 ± 2.1 | 1.7 ± 1.9 | 4.3 ± 2.5 | <0.001 |
| Heart rate (bpm) | 86.2 ± 17.1 | 86.0 ± 17.1 | 87.8 ± 15.0 | 0.42 |
| Systolic blood pressure | 125.3 ± 20.7 | 126.3 ± 21.0 | 118.7 ± 17.0 | 0.02 |
| (mmHg) | | | | |
| Diastolic blood | 72.2 ± 14.5 | 72.5 ± 15.0 | 70.4 ± 10.5 | 0.37 |
| pressure (mmHg) | | | | |
| Respiratory rate (bpm) | 17.2 ± 6.2 | 17.0 ± 6.3 | 18.6 ± 5.6 | 0.09 |
| Oxygen saturation (%) | 96.6 ± 2.6 | 96.6 ± 2.6 | 96.4 ± 2.3 | 0.39 |
| Supplemental oxygen | 26 | 24 | 44 | 0.01 |
| (%) | | | | |

In normal volunteers, mean EtCO₂ was 36.3±2.8 mmHg (95% CI 35.1-37.4, Table 3). Data are presented as mean±SD, n=24. There were no significant differences among the five measured breaths each day or among the mean EtCO₂s in an individual over the three separate days. Age and gender did not affect EtCO₂.

TABLE 3
EtCO₂ in normal individuals over 5 separate days

| | | |
|---|---|---|
| Age (yrs) | 40.0 ± 12.0 | |
| Female no. | 14 | |
| Smoking no. | | |
| Never | 20 | |
| Past | 4 | |
| Current | 0 | |
| EtCO₂ by breath (Day 1) (mmHg) | | p = 0.21 |
| Breath 1 | 36.7 ± 3.0 | |
| Breath 2 | 36.3 ± 2.9 | |
| Breath 3 | 36.7 ± 3.0 | |
| Breath 4 | 37.1 ± 3.5 | |
| Breath 5 | 37.3 ± 3.6 | |
| EtCO₂ by day (mmHg) | | p = 0.25 |
| Day 1 | 36.6 ± 3.0 | |
| Day 2 | 36.6 ± 3.8 | |
| Day 3 | 35.6 ± 3.6 | |
| Overall mean EtCO₂ (mmHg) | 36.4 ± 2.8 | |

There was no significant difference in EtCO₂ between normal controls and the no PE group (36.3±2.8 mmHg vs. 35.5±6.8 mmHg respectively, p=0.56, FIG. 3). The group with PE had a significantly lower EtCO₂ (30.5±5.5 mmHg, vs. healthy volunteers p<0.001), which was also significant compared with the no PE group (P<0.001). Mean EtCO₂ was not different in the two D-dimer groups (35.3±5.9 mmHg D-dimer positive vs. 36.1±5.2 in D-dimer negative groups, p=0.35). There were no adverse events related to EtCO₂ measurement.

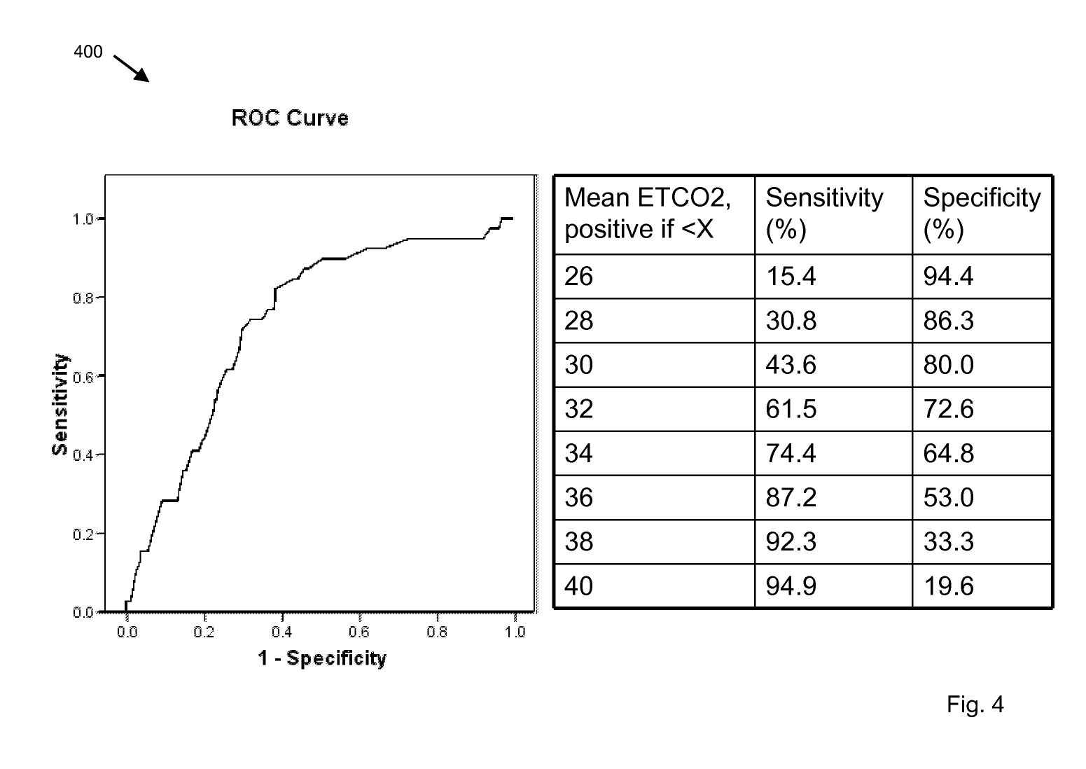

A receiver operator characteristics (ROC) curve demonstrating the ability of EtCO₂ to discriminate between patients with and without PE and the corresponding sensitivities and specificities to a given EtCO₂ measurement are shown in FIG. 4 (AUC=0.739). In order to avoid the most unnecessary procedures in the diagnosis of PE while maintaining optimal sensitivity for diagnosis, a cut off of 36 mmHg was chosen for further analysis of the characteristics of this test. At this cut off, the negative predictive value was 96.6% (95% CI 92.3-98.5, Table 4).

TABLE 4
Test performance characteristics

| | | | Positive | Negative |
|---|---|---|---|---|
| | | | Predictive | Predictive |
| | Sensitivity | Specificity | Value | Value |
| | (%, 95% CI) | (%, 95% CI) | (%, 95% CI) | (%, 95% CI) |
| EtCO₂ <36 All | 87.2 | 53.0 | 21.1 | 96.6 |
| Comers | (73.3-94.4) | (47.0-58.8) | (15.5-28.1) | (92.3-98.5) |
| EtCO₂ <36, | 91.9 | 49.0 | 21.1 | 97.6 |
| excluding >44 | (78.7-97.2) | (42.8-55.2) | (15.5-28.1) | (93.2-99.2) |
| Wells Score | 61.5 | 83.3 | 34.8 | 93.8 |
| ≧4 | (45.9-75.1) | (78.4-87.3) | (24.6-46.6) | (89.9-96.2) |
| EtCO₂ <36 All | 92.3 | 45.2 | 19.6 | 97.6 |
| Comers + | (79.7-97.3) | (39.4-51.1) | (14.5-25.9) | (93.2-99.2) |
| Wells Score | | | | |
| ≧4 | | | | |

When patients with EtCO₂≧36 mmHg but <44 mmHg (2.78 SD above normal) were analyzed, there was an increase in negative predictive value to 97.6% (95% CI 93.2-99.2). A negative predictive value for Wells score <4 of 93.8% (95% CI 89.9-96.2) was found in this population. In combining the Wells score <4 with the EtCO₂≧36 mmHg without restriction on maximum EtCO₂, the negative predictive value again rose to 97.6% (95% CI 93.2-99.2).

In this study, it was shown that a safe, simple, inexpensive, bedside test for EtCO₂ has a high negative predictive value in excluding PE and that the EtCO₂ measured with the oral capnometer 102 of the invention in combination with the Wells Score improves negative predictive value to a very high level of accuracy.

Dead space fraction (Vd/Vt), measured by comparing total exhaled partial pressure CO₂ (pCO₂) with arterial partial pressure CO₂ (paCO₂), has previously been shown to be abnormal in pulmonary embolism and Vd/Vt in combination with D-dimer testing is effective at ruling out PE, as described for example in Kline referenced above, Verschuren F, Liistro G, Coffeng R, Thys F, Roeseler J, Zech F, Reynaert M., Volumetric capnography as a screening test for pulmonary embolism in the emergency department, Chest 2004: 125(3): 841-850, Robin E D, Julian D G, Travis D M, Crump C H., A physiologic approach to the diagnosis of acute pulmonary embolism, N Engl J Med 1959: 260(12): 586-591, and Anderson D R, Kovacs M J, Dennie C, Kovacs G, Stiell I, Dreyer J, McCarron B, Pleasance S, Burton E, Cartier Y, Wells P S., Use of spiral computed tomography contrast angiography and ultrasonography to exclude the diagnosis of pulmonary embolism in the emergency department, J Emerg Med 2005: 29(4): 399-404 each of which is incorporated herein by reference in its entirety. However, the requirement of specialized equipment and an arterial puncture limit its widespread adaptation. EtCO₂ measured only with the oral capnometer 102 is a surrogate for dead space measurement.

Various cut off levels of EtCO₂ were examined to determine optimal sensitivity and specificity of this test. Using a cut off of ≧36 mmHg, a negative predictive value of 96.6% was achieved, which is similar to that reported with d-dimer testing as described for example in Stein referenced above. There was a small improvement after excluding patients with an EtCO₂ significantly outside of the range of normal, but might confuse clinical decision-making without a concomitantly large improvement in test characteristics. The addition of the Wells score <4 to the EtCO₂ measurement similarly numerically improved the testing characteristics without adding further confusion about patient exclusions. It was found that at the lower levels of EtCO₂, there was a substantial increase in specificity for PE. This improved specificity at lower EtCO₂ levels is in marked contrast with D-dimer, with results that are either positive or negative.

In the study group, 166 subjects had an EtCO₂>36 mmHg and would not have undergone further testing if that were used as the sole criterion for ruling out PE. Of these 166 subjects, 20 had a Wells score of 4.0 or higher. Thus, in the study, 146/298 (49%) of subjects would have been spared further evaluation for PE using these criteria. Three of 39 PEs would be missed in the study using these criteria. All three of these patients were discovered to have hypoventilation after further evaluation during the hospitalization (morbid obesity, chronic narcotic use and interstitial lung disease).

The importance of sparing these diagnostic procedures is not trivial. In the cohort, 226 patients (76%) underwent diagnostic CT scanning. The long-term risks of exposure to radiation from chest CT scanning are a concern, as described for example in Brenner and Amis described above and Strzelczyk J J, Damilakis J, Marx M V, Macura K J., Facts and controversies about radiation exposure, part 2: low-level exposures and cancer risk, J Am Coll Radiol 2007: 4(1): 32-39 and Strzelczyk J J, Damilakis J, Marx M V, Macura K J., Facts and controversies about radiation exposure, part 1: controlling unnecessary radiation exposures, J Am Coll Radiol 2006: 3(12): 924-931 each of which is incorporated herein by reference in its entirety. The typical contrast-enhanced chest CT for pulmonary embolism evaluation delivers approximately 20 mSv of radiation, as described for example in Brenner referenced above and Coche E, Vynckier S, Octave-Prignot M., Pulmonary embolism: radiation dose with multi-detector row CT and digital angiography for diagnosis, Radiology 2006: 240(3): 690-697 which is incorporated herein by reference in its entirety. This dose from a single CT approaches the 40 mSv widely thought of as a dangerous limit from historical data, as described for example in Brenner, Strzelczyk, and Coche referenced above. In this study alone, five people were enrolled twice in the six-month study. While there is debate about the “safe limit” of radiation exposure, the American College of Radiology has called for controlling unnecessary radiation exposure, as described for example in Strzelczyk referenced above. The monetary savings from preventing unnecessary CT studies is also potentially substantial. For example, at a cost per study of $1739, as described for example in Stein P D, Woodard P K, Weg J G, Wakefield T W, Tapson V F, Sostman H D, Sos T A, Quinn D A, Leeper K V, Jr., Hull R D, Hales C A, Gottschalk A, Goodman L R, Fowler S E, Buckley J D., Diagnostic pathways in acute pulmonary embolism: recommendations of the PIOPED II Investigators, Radiology 2007: 242(1): 15-21 which is incorporated herein by reference in its entirety, patients in the study underwent a total of 226 contrast enhanced helical CTs, 120 of which could potentially be spared saving $208,680. The study included both inpatients and patients in the Emergency Department to capture the complete population perceived to be at risk for PE. Because patients who underwent only D-dimer testing were not included, the pre-test probability for PE in the cohort may have been increased. Despite this potential bias, EtCO₂ was similar in the normal controls and the group without PE, suggesting that physiologically the group without PE was similar to normals. Too few patients had PEs in the group with D-dimer data to allow a meaningful direct comparison with EtCO₂. While the CT positivity rate for PE was lower than some prior published reports, as described for example in Perrier and Stein referenced above and van Belle A, Buller H R, Huisman M V, Huisman P M, Kaasjager K, Kamphuisen P W, Kramer M H, Kruip M J, Kwakkel-van Erp J M, Leebeek F W, Nijkeuter M, Prins M H, Sohne M, Tick L W., Effectiveness of managing suspected pulmonary embolism using an algorithm combining clinical probability, D-dimer testing, and computed tomography. JAMA 2006: 295(2): 172-179 which is incorporated herein by reference in its entirety, it is similar to other publications in the literature and may represent local practice patterns, as described for example in Anderson referenced above and Yap K S, Kalff V, Turlakow A, Kelly M J., A prospective reassessment of the utility of the Wells score in identifying pulmonary embolism, Med J Aust 2007: 187(6): 333-336 which is incorporated herein by reference in its entirety. The EtCO₂ would likely be abnormal in conditions affecting metabolic activity or carbon dioxide excretion such as pregnancy, end-stage chronic obstructive lung disease or advanced neuromuscular disease; therefore patients known to have these conditions from participation were excluded, totaling fewer than 10 patients. Thyroid disease at its extremes may affect EtCO₂ results, but this is often not known at initial evaluation, thus these patients were not excluded. EtCO₂ cannot distinguish between type of pulmonary arterial obstruction such as acute PE, chronic thromboembolic disease or tumor emboli. No CT angiograms showed changes typical for chronic thromboembolic pulmonary hypertension.

Thus, a cheap, simple, readily available, non-invasive test of EtCO₂ combined with a bedside prediction tool may be useful to exclude pulmonary embolism in patients without pregnancy or advanced lung or neuromuscular disease.

Accurate measurement of orally exhaled gases may be useful additionally in a pediatric population, with patients under sedation, with patients who have been intubated, to measure expired gases continuously, and the like.

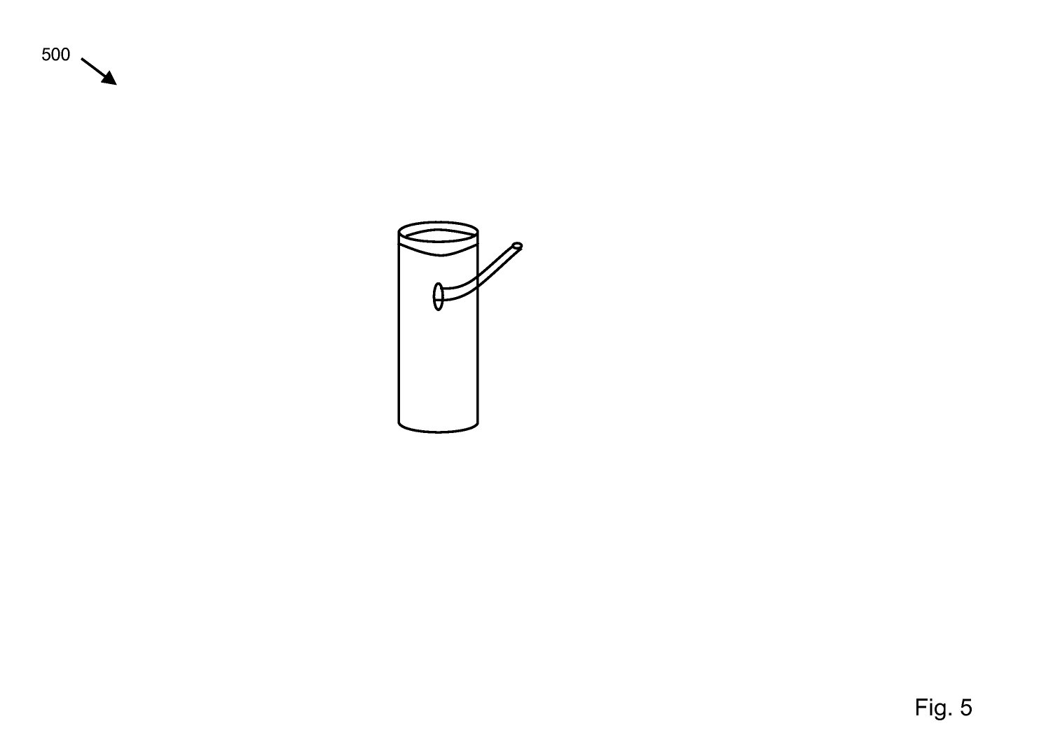

The oral capnometer 102 of the invention may be constructed by adapting the sampling input of a capnometer, as shown in FIG. 1, with an oral adaptor. For example, the oral adaptor may be a hollow-bodied oral gas capture member that sits in a subject's mouth, having formed in the member an aperture through which a subject may exhale gases and an aperture for placement of a sampling tube of the capnometer that positions the sampling tube within the capture member and allows exhaled gases to enter the sampling tube. In embodiments, the adaptor may be of any shape and may bear any markings. For example, as in FIG. 5, the sampling tube may be placed through a hole in the sidewall of a hollow tube. In an embodiment, the tube may have dimensions of 1.5 cm diameter×5 cm length. The sampling tube may be formed from flexible, plastic tubing. The oral gas capture member may be formed from any suitable material, such as plastic, metal, glass, or the like. In an embodiment, the oral gas capture member may be disposable.

The oral capnometer 102 may be used to construct a capnograph by measuring carbon dioxide levels over time.

The oral capnometer 102 may be useful in measuring carbon dioxide levels in order to estimate cardiac output and metabolism; diagnose hypoventilation, bronchitis, emphysema, asthma, congenital heart disease, hypothermia, diabetes, circulatory shock; and obtain information about the effectiveness of CPR and the return of spontaneous circulation (ROSC), CO₂ production, pulmonary (lung) perfusion, alveolar ventilation, respiratory patterns, and elimination of CO₂ from the anesthesia breathing circuit and ventilator.

In an embodiment, evaluating pulmonary embolism in a subject may include measuring end tidal partial pressure of exhaled carbon dioxide in the subject, wherein the measurement is made orally, obtaining a clinical approximation of dead space ventilation based on the measurement, and excluding pulmonary embolism when the end tidal partial pressure of exhaled carbon dioxide reaches a threshold. The threshold may be at least 36 mm Hg. The evaluation may further include applying a clinical prediction rule. The rule may include calculating a Wells score, and pulmonary embolism may be excluded when the Wells score is at least four. The subject may be a pediatric subject, sedated, intubated, and the like.

In an embodiment, an oral capnometer 102 may include an oral gas capture member 104, 602, 702, for collecting expired gases from the mouth, and a carbon dioxide measuring device attached to the oral gas capture member 104, 602, 702 for determining levels of expired carbon dioxide from the mouth of a subject. The subject may be a pediatric subject, sedated, intubated, and the like. Carbon dioxide levels may be measured continuously. The expired carbon dioxide may be end tidal carbon dioxide.

In an embodiment, a method of measuring end tidal carbon dioxide in a subject may include collecting expired gases from the mouth through an oral gas capture member 104, 702 adapted to be disposed on the sampling input of a carbon dioxide measuring device and determining levels of expired carbon dioxide in the expired gas. In another embodiment, a method of measuring end tidal carbon dioxide in a subject may include a carbon dioxide measuring device that directly collects expired gases from the mouth of the subject by means of an integral gas capture chamber 602. The subject may be a pediatric subject, sedated, intubated, awake, spontaneously breathing, and the like. Carbon dioxide levels may be measured continuously. The expired carbon dioxide may be end tidal carbon dioxide.

In an embodiment, an oral capnometer 102 may include an oral gas capture member 602 for collecting expired gases from the mouth of a subject; a gas sensor for identifying and measuring at least one exhaled gas; and a housing for housing the gas sensor, wherein the housing is integral with the oral gas capture member 602. The exhaled gas may be at least one of carbon dioxide, carbon monoxide, nitrogen, oxygen, and ketone. The subject may be at least one of awake, spontaneously breathing, pediatric, sedated, intubated, sleeping, and the like. Gas levels may be measured continuously. The expired carbon dioxide may be end tidal carbon dioxide. The gas sensor may also the measure pH of an exhaled gas.

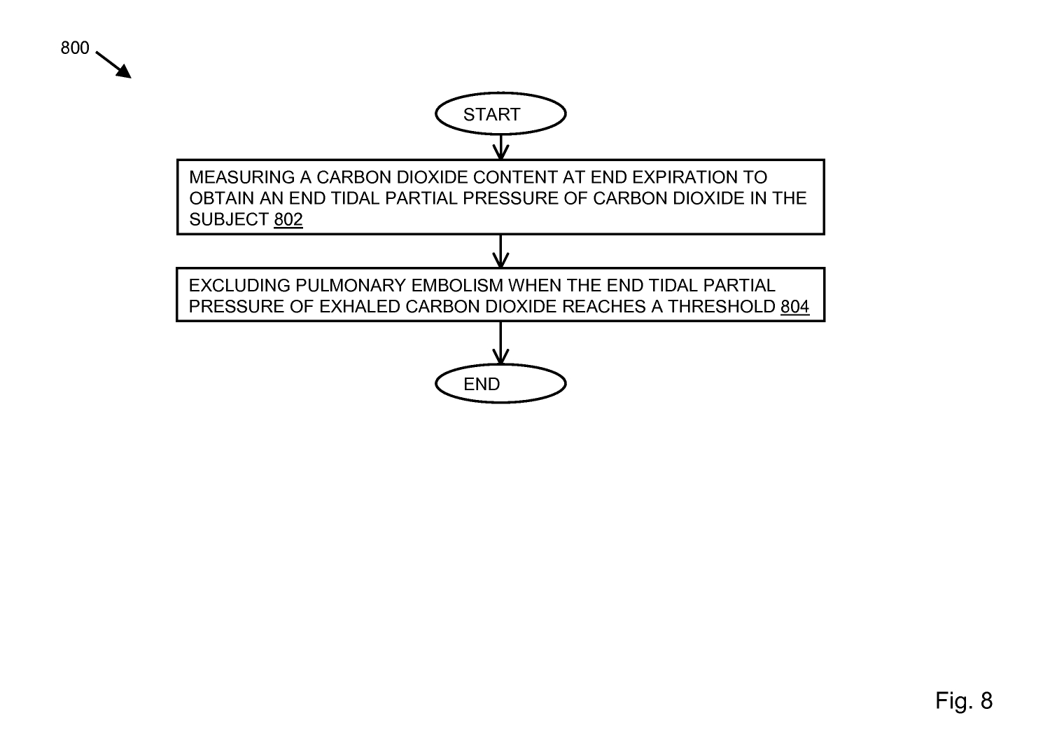

Referring to FIG. 8, a method of evaluating pulmonary embolism in a subject may include measuring a carbon dioxide content at end expiration to obtain an end tidal partial pressure of carbon dioxide in the subject 802 and excluding pulmonary embolism when the end tidal partial pressure of exhaled carbon dioxide reaches a threshold 804. The measurement may be made orally. A clinical approximation of dead space ventilation is based on the measurement. The threshold may be at least 36 mm Hg. The method of evaluating pulmonary embolism may further include applying a clinical prediction rule. The rule may include calculating a Wells score. Pulmonary embolism is excluded when the Wells score is at least four. The subject may be at least one of sedated, intubated, and pediatric.

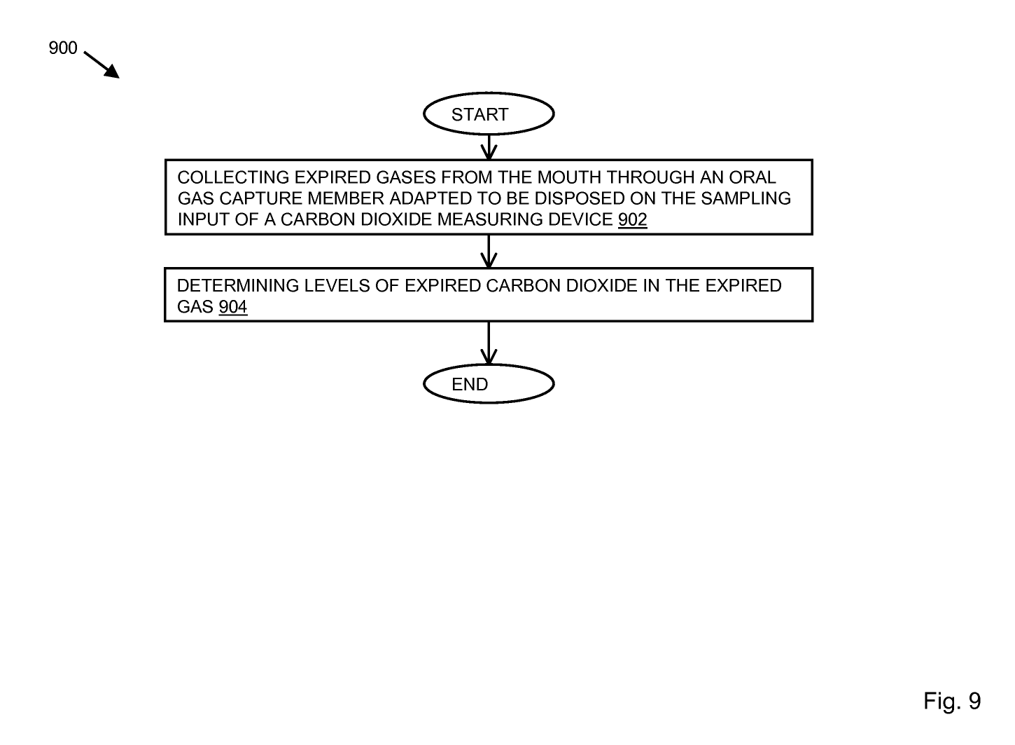

Referring to FIG. 9, a method of measuring end tidal carbon dioxide in a subject may include collecting expired gases from the mouth through an oral gas capture member adapted to be disposed on the sampling input of a carbon dioxide measuring device 902 and determining levels of expired carbon dioxide in the expired gas 904. The subject is at least one of sedated, intubated, and pediatric. The carbon dioxide levels may be measured continuously. The expired carbon dioxide may be end tidal carbon dioxide.

The methods and systems described herein may be deployed in part or in whole through a machine that executes computer software, program codes, and/or instructions on a processor. The processor may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer to peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipments, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

All documents referenced herein are hereby incorporated by reference.

What is claimed is:

1. A method of evaluating pulmonary embolism in a subject comprising:
measuring a carbon dioxide content at end expiration to obtain an end tidal partial pressure of exhaled carbon dioxide in the subject to provide a measurement, wherein the measurement is made orally, wherein a clinical approximation of dead space ventilation is based on the measurement; and
excluding pulmonary embolism when the end tidal partial pressure of exhaled carbon dioxide reaches a threshold.

2. The method of claim 1 wherein the threshold is at least 36 mm Hg.

3. The method of claim 1 further comprising applying a clinical prediction rule.

4. The method of claim 3 wherein applying the clinical prediction rule includes calculating a Wells score, and wherein pulmonary embolism is excluded when the Wells score is at least four.

5. The method of claim 1, wherein the subject is at least one of sedated, intubated, and pediatric.

6. An oral capnometer comprising:
an oral gas capture member that collects expired gases from a mouth of a subject; and
a carbon dioxide measuring device attached to the oral gas capture member that determines levels of carbon dioxide at end expiration from the mouth of the subject.

7. The oral capnometer of claim 6 wherein the subject is at least one of sedated, intubated, and pediatric.

8. The oral capnometer of claim 6, wherein the levels of carbon dioxide are measured continuously.

9. The oral capnometer of claim 6 wherein the levels of carbon dioxide at end expiration include end tidal carbon dioxide.

10. A method of measuring end tidal carbon dioxide in a subject comprising:
collecting expired gas from a mouth of the subject through an oral gas capture member adapted to be disposed on a sampling input of a carbon dioxide measuring device; and
determining levels of expired carbon dioxide in the expired gas.

11. The method of claim 10 wherein the subject is at least one of sedated, intubated, and pediatric.

12. The method of claim 10 wherein the expired carbon dioxide is determined continuously.

13. The method of claim 10 wherein the expired carbon dioxide is end tidal carbon dioxide.

14. An oral capnometer comprising:
an oral gas capture member that collects expired gases from a mouth of a subject; and
a carbon dioxide measuring device integral with the oral gas capture member for determining levels of expired carbon dioxide from the mouth of the subject.

15. The oral capnometer of claim 14 wherein the subject is at least one of sedated, intubated, and pediatric.

16. The oral capnometer of claim 14 wherein the levels of expired carbon dioxide are determined continuously.

17. The oral capnometer of claim 14 wherein the expired carbon dioxide is end tidal carbon dioxide.

18. A system comprising:
an oral gas capture member that collects expired gases from a mouth of a subject;
a gas sensor that identifies and measures an exhaled gas; and
a housing that houses the gas sensor, wherein the housing is integral with the oral gas capture member.

19. The system of claim 18 wherein the exhaled gas includes one or more of carbon dioxide, carbon monoxide, nitrogen, oxygen, and ketone.

20. The system of claim 19 wherein the gas sensor measures a pH of the exhaled gas.
